(19) United States
(12) Patent Application Publication
(10) Pub. No.: US 2008/0269525 A1
(43) Pub. Date: Oct. 30, 2008

(54) SYNTHESIS OF STABLE CYCLOPROPENYLIDENES

(75) Inventors: Guy Bertrand, Riverside, CA (US); Yves Canac, Castanet Tolosan (FR); Vincent Lavallo, Riverside, CA (US); Bruno Donnadieu, Riverside, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 12/101,100

(22) Filed: Apr. 10, 2008

Related U.S. Application Data
(60) Provisional application No. 60/911,609, filed on Apr. 13, 2007.

(51) Int. Cl. C07C 211/40

(52) U.S. Cl. 564/461

(57) ABSTRACT

The invention provides for stable and isolable cyclopropenylidenes of Formula I and stable and isolable cyclopropenylidene-metal complexes of Formula II, and methods of making thereof.

### CROSS-REFERENCES TO RELATED APPLICATIONS

This application claims the benefit of Provisional Application Ser. No. 60/911,609, filed Apr. 13, 2007, the content of which is incorporated herein by reference.

### STATEMENT AS TO RIGHTS TO INVENTIONS MADE UNDER FEDERALLY SPONSORED RESEARCH AND DEVELOPMENT

This invention was made with Government support under Grant No. GM068225, awarded by the National Institutes of Health. The Government has certain rights in this invention.

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Carbenes are compounds with a neutral dicoordinate carbon atom, which features either two singly occupied nonbonding orbitals (a triplet state) or a lone pair and an accessible vacant orbital (a singlet state). With only six electrons in its valence shell, the carbene center defies the octet rule, and for a long time carbenes have been considered as prototypical reactive intermediates (a, b). During the past two decades, carbene chemistry has undergone a profound revolution. Persistent triplet carbenes have been observed (c), and singlet carbenes have been isolated (d-f) and even become powerful tools for synthetic chemists (g, h). However, it is generally believed that singlet carbenes can be isolated only if their electron deficiency is reduced by the presence of at least one π-donor heteroatom directly bonded to the carbene center (i).

Cyclopropenylidene (C₃H₂) A is a cyclic singlet carbene (see, FIG. 1), Since its first radio astronomical detection in 1985 (j), it has been inferred to be the most abundant cyclic hydrocarbon observed in interstellar space (k). It is detectable in molecular clouds, circumstellar shells, and at least one external galaxy. Cyclopropenylidene A and its linear isomers B and C (see, FIG. 1) have also been observed in several hydrocarbon-rich flames and may be involved in the chemistry of soot formation (l). Although carbenes are very reactive, they are not necessarily prone to self-rearrangement, which accounts for their stability in the near-vacuum of deep space and in the low-density medium of flame.

However, the current literature suggested that cyclopropenylidene A and its derivatives are highly unstable in condensed phases. For example, Reisenauer et al. (m) were able to detect cyclopropenylidene A by infrared spectroscopy in a solid argon matrix, but it survives for only a few hours at 35-40 K and then it polymerized. Other researchers have attempted to prepare and isolate bis(dialkylamino)cyclopropenylidenes, such as 2 (see FIG. 2), without success (n-p). The transient existence of bis(diisopropylamino)cyclopropenylidene 2 has been postulated based on chemical trapping experiments, but it has been described as a highly unstable molecule, defying isolation or even observation in the free state (n-t). For example, Yoshida et al. and Weiss et al. independently attempted to prepare 2, however, in spite of their efforts, they were only able to prepare a lithium adduct 4, which they described to be unstable and not isolable (n-q).

Thus, in view of the above, there exists a need in the art for cyclopropenylidenes and cyclopropenylidene-metal complexes that are stable and isolable. The present invention fulfills this and other needs.

### REFERENCES CITED

a. R. A. Moss, M. S. Platz, M. Jones Jr., Eds., Reactive Intermediate Chemistry (Wiley, New York, 2004).
b. U. H. Brinker, Ed., Advances in Carbene Chemistry (JAI, Greenwich, Conn., 2001).
c. T. Itoh, Y. Nakata, K. Hirai, H. Tomioka, J. Am. Chem. Soc. 128, 957 (2006).
d. F. E. Hahn, Angew. Chem. Int. Ed. Engl. 45, 1348 (2006).
e. R. W. Alder et al., Angew. Chem. Int. Ed. Engl. 43, 5896 (2004).
f. D. Bourissou, O. Guerret, F. P. Gabbai, G. Bertrand, Chem. Rev. 100, 39 (2000).
g. E. Peris, R. H. Crabtree, Coord. Chem. Rev. 248, 2239 (2004).
h. N. M. Scott, S. P. Nolan, Eur. J. Inorg. Chem. 2005, 1815 (2005).
i. W. Kirmse, Angew. Chem. Int. Ed. Engl. 43, 1767 (2004).
j. P. Thaddeus, J. M. Vrtilek, C. A. Gottlieb, Astrophys. J. 299, 163 (1985).
k. R. I. Kaiser, Chem. Rev. 102, 1309 (2002).
l. C. A. Taatjes et al., Phys. Chem. Chem. Phys. 7, 806 (2005).
m. H. P. Reisenauer, G. Maier, A. Riemann, R. W. Hoffmann, Angew. Chem. Int. Ed. Engl. 23, 641 (1984).
n. Z. Yoshida, Pure Appl. Chem. 54, 1059 (1982).
o. R. Weiss, C. Priesner, H. Wolf, Angew. Chem. Int. Ed. Engl. 17, 446 (1978).
p. R. Weiss, M. Hertel, H. Wolf, Angew. Chem. Int. Ed. Engl. 18, 473 (1979).
q. M. Tamm, A. Grzegorzewski, F. E. Hahn, J. Organomet. Chem. 501, 309 (1995).
r. H. Schumann et al., Angew. Chem. Int. Ed. Engl. 36, 2232 (1997).
s. Cyclopropenylidene transition metal complexes have been known since 1968 (t). and main group adducts were recently reported (r).
t. K. Ofete, Angew. Chem. Int. Ed. Engl. 7, 950 (1968).

### BRIEF SUMMARY OF THE INVENTION

In one aspect, the present invention provides for novel, isolable, stable cyclopropenylidenes compounds of Formula I:

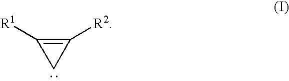

In Formula I, R¹ and R² are each members independently selected from the group consisting of amino, aryl, heteroaryl, C₁₋₁₀ alkoxy, C₂₋₁₀ alkenyloxy, C₂₋₁₀ alkynyloxy, C₁₋₁₀ alkyl, C₃₋₁₀ cycloalkyl, C₂₋₁₀ alkenyl, C₃₋₁₀ cycloalkenyl, C₂₋₁₀ alkynyl, halogen, aryloxy, heteroaryloxy, C₂₋₁₀ alkoxycarbonyl, C₁₋₁₀ alkylthio, C₂₋₁₀ alkenylthio, C₂₋₁₀ alkynylthio, C₁₋₁₀ alkylsulfonyl, C₁₋₁₀ alkylsulfinyl, aryl-C₁₋₁₀ alkyl, heteroaryl-C₁₋₁₀ alkyl, aryl-C₁₀ heteroalkyl, heteroaryl-C₁₋₁₀ heteroalkyl, a phosphorus group, a silicon group and a boron group; wherein R¹ and R² are optionally combined to form a 5- to 8-membered carbocyclic or heterocyclic ring. The aliphatic or aromatic portions of R¹ and R² are optionally substituted with from 1 to 4 substituents selected from the group consisting of halogen, cyano, nitro, C₁₋₄ alkyl, C₂₋₆ alkenyl, C₂₋₆ alkynyl, aryl, C₁₋₆ alkoxy, C₂₋₆ alkenyloxy, C₂₋₆ alkynyloxy, aryloxy, C₂₋₆ alkoxycarbonyl, C₁₋₆ alkylthio, C₁₋₆ alkylsulfonyl, C₁₋₆ alkylsulfinyl, oxo, imino, thiono, primary amino, carboxyl, C₁₋₆ alkylamino, C₁₋₆ dialkylamino, amido, nitrogen heterocycles, hydroxy, thiol and phosphorus groups.

In another aspect, the present invention provides for stable, isolable cyclopropenylidene-metal complexes, wherein the metal is selected from group 1, 2-4, 6-9, 11-12 and 14-16 of the periodic table metal, and the cyclopropenylidene is of Formula I or Ia.

In another aspect, the present invention provides for stable and isolable cyclopropenylidene metal complexes of Formula II

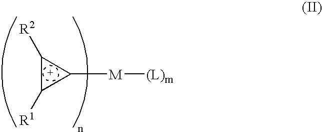

wherein R¹ and R² are as described for Formula I; the subscript n is an integer from 1 to 8; L represents an anionic, neutral or electron-donating ligand; the subscript m is an integer from 0 to 6; and M represents a group 1, 2, or 11 metal ion. The present invention also provides methods of preparing stable and isolable cyclopropenylidenes of Formula I and cyclopropenylidene-metal complexes of Formula II.

### BRIEF DESCRIPTION OF THE DRAWINGS

FIG. 1 shows the structure of a cyclopropenylidene (A) and its linear isomers (B) and (C).

FIG. 2 shows the structure of bis(diisopropylamino)cyclopropenylidene 2 and its lithium adduct 4.

FIG. 3 shows the molecular ORTEP view of cyclopropenium tetraphenylborate 1 and cyclopropenylidene 2. For clarity, the tetraphenylborate anion and hydrogen atoms are omitted in the left and bottom images, respectively.

FIG. 4A shows the molecular ORTEP view of the BF₄⁻ salt of cyclopropenylidene-lithium adduct 4 in the solid state. (Atom color code: C black; N blue; Li pink, B yellow, F green) The isopropyl groups on the nitrogen have been omitted for clarity. FIG. 4B shows the molecular ORTEP view a single cyclopropenylidene-lithium adduct 4. The hydrogen atoms on the i-propyl groups have been omitted for clarity.

FIG. 5 illustrates the X-ray crystal structure of bis(diisopropylaminocyclopropenylidene)-magnesium complex 6.

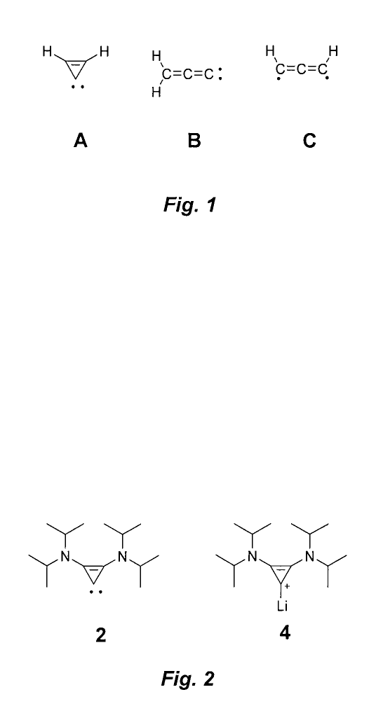

### DETAILED DESCRIPTION OF THE INVENTION

### Abbreviations and Definitions

Abbreviations used herein have their common and accepted meanings to one of skill in the art. Examples of the abbreviations are t-Bu, tertiary butyl; Me, methyl; THF, tetrahydrofuran.

In the present description the term “alkyl”, alone or in combination, refers to a straight-chain or branched-chain alkyl group having the indicated number of carbon atoms. For example, C₁₋₁₀ alkyl refers to an alkyl group having from one to ten carbon atoms with the remaining valences occupied by hydrogen atoms. Preferred alkyl groups are those with 1 to 8 carbon atoms, more preferably a straight or branched-chain alkyl group with 1 to 6 carbon atoms and particularly preferred are straight or branched-chain alkyl groups with 1 to 4 carbon atoms. Examples of straight-chain and branched C₁₋₁₀ alkyl groups are methyl, ethyl, propyl, isopropyl, butyl, isobutyl, tert-butyl, the isomeric pentyls, the isomeric hexyls, the isomeric heptyls and the like.

The term “cycloalkyl”, alone or in combination, refers to a cyclic alkyl group having 3 to 8 carbon atoms as ring vertices. Preferred cycloalkyl groups are those having 3 to 6 carbon atoms. Examples of C₃₋₈ cycloalkyl are cyclopropyl, methyl-cyclopropyl, dimethylcyclopropyl, cyclobutyl, methyl-cyclobutyl, cyclopentyl, methyl-cyclopentyl, cyclohexyl, methyl-cyclohexyl, dimethyl-cyclohexyl, cycloheptyl and cyclooctyl.

The term “cycloalkenyl”, alone or in combination, refers to a cyclic alkenyl group having 3 to 8 carbon atoms as ring vertices. Preferred cycloalkyl groups are those having 3 to 6 carbon atoms. Examples of C₃₋₈ cycloalkyl are cyclopropenyl, cyclopentenyl dimethylcyclopropenyl and cyclobutyl.

The term “alkoxy”, alone or in combination, signifies a group of the formula alkyl-O— in which the term “alkyl” has the previously given definition. Examples of alkoxy group include methoxy, ethoxy, n-propoxy, iso-propoxy, n-butoxy, iso-butoxy, sec-butoxy and tert-butoxy. Preferred alkoxy groups are methoxy and ethoxy.

The term “alkenyl”, alone or in combination refers to a straight-chain, cyclic or branched hydrocarbon residue comprising at least one olefinic bond and the indicated number of carbon atoms. Preferred alkenyl groups have up to 8, preferably up to 6, particularly preferred up to 4 carbon atoms. Examples of alkenyl groups are ethenyl, 1-propenyl, 2-propenyl, isopropenyl, 1-butenyl, 2-butenyl, 3-butenyl, isobutenyl, 1-cyclohexenyl, 1-cyclopentenyl.

The term “alkynyl”, alone or in combination refers to a straight-chain or branched hydrocarbon residue having a carbon triple bond and the indicated number of carbon atoms. Preferred alkynyl groups have up to 8, preferably up to 6, particularly preferred up to 4 carbon atoms. Examples of alkynyl groups are ethynyl, 1-propynyl, 1-butynyl and 2-butynyl

The terms “alkylthio,” “alkylsulfonyl,” “alkylsulfinyl” and “arylsulfonyl” refer to groups having the formula —S—Rⁱ—S(O)₂—Rⁱ, —S(O)—Rⁱ and —S(O)₂R^{j}, respectively, in which R^{j} is an alkyl group as previously defined and R^{j} is an aryl group as previously defined.

The terms “alkenyloxy” and “alkynyloxy” refer to groups having the formula —O—Rⁱ in which Rⁱ is an alkenyl or alkynyl group, respectively.

The terms “alkenylthio” and “alkynylthio” refer to groups having the formula —S—R^{k} in which R^{k} is an alkenyl or alkynyl group, respectively.

The term “alkoxycarbonyl” refers to a group having the formula —C(O)O—Rⁱ, wherein Rⁱ is an alkyl group as defined above and wherein the total number of carbon atoms refers to the combined alkyl and carbonyl moieties.

The term “aryl” means, unless otherwise stated, a polyunsaturated, typically aromatic, hydrocarbon group which can be a single ring or multiple rings (up to three rings) which are fused together or linked covalently, and which optionally carries one or more substituents, for example, such as halogen, trifluoromethyl, amino, alkyl, alkoxy, alkylcarbonyl, cyano, carbamoyl, alkoxycarbamoyl, methylendioxy, carboxy, alkoxycarbonyl, aminocarbonyl, alkyaminocarbonyl, dialkylaminocarbonyl, hydroxy, nitro and the like. Non-limiting examples of unsubstituted aryl groups include phenyl, naphthyl and biphenyl. Examples of substituted aryl groups include, but are not limited to, phenyl, chlorophenyl, trifluoromethylphenyl, chlorofluorophenyl and aminophenyl.

The term “heteroalkyl,” by itself or in combination with another term, means, unless otherwise stated, a stable straight or branched chain, or combinations thereof, consisting of the stated number of carbon atoms and from one to five heteroatoms, more preferably from one to three heteroatoms, selected from the group consisting of O, N, Si and S, and wherein the nitrogen and sulfur atoms may optionally be oxidized and the nitrogen heteroatom may optionally be quaternized. The heteroalkyl group is attached to the remainder of the molecule through a carbon atom or a heteroatom.

The term “heterocycloalkyl” by itself or in combination with another term refers to a cyclic hydrocarbon radical or a combination of a cyclic hydrocarbon radical with a straight or branched chain alkyl group, consisting of the stated number of carbon atoms and from one to three heteroatoms as ring members selected from the group consisting of O, N, Si and S, and wherein the nitrogen and sulfur atoms may optionally be oxidized and the nitrogen heteroatom may optionally be quaternized. The heterocycloalkyl group is attached to the remainder of the molecule through a carbon atom or a heteroatom.

The term “heteroaryl”, alone or in combination, unless otherwise stated, signifies aromatic 5- to 10-membered heterocycle which contains one or more, preferably one or two hetero atoms selected from nitrogen, oxygen and sulfur, wherein nitrogen or oxygen are preferred. If desired, it can be substituted on one or more carbon atoms substituents such as halogen, alkyl, alkoxy, cyano, haloalkyl, preferably trifluoromethyl, and heterocyclyl, preferably morpholinyl or pyrrolidinyl, and the like. Examples of heteroaryls include, but are not limited to, pyridinyl or furanyl.

The term “heterocycle,” alone or in combination, unless otherwise stated, refers to heteroaryl and heterocycloalkyl groups.

The term “aryloxy” and “heteroaryloxy”, alone or in combination, signifies a group of the formula aryl-O— and heteroaryl-O—, respectively, in which the terms “aryl” and “heteroaryl” have the significance as provided above, such as phenyloxy, and pyridyloxy, and the like.

The term “amino”, alone or in combination, signifies a primary, secondary or tertiary amino group bonded to the remainder of the molecule via the nitrogen atom, with the secondary amino group carrying an alkyl, cycloalkyl, alkenyl, cycloalkenyl, alkynyl, heteroalkyl, heterocycloalkyl, aryl or heteroaryl substituent and the tertiary amino group carrying two similar or different alkyl, cycloalkyl, alkenyl, cycloalkenyl, alkynyl, heteroalkyl, heterocycloalkyl, aryl or heteroaryl substituents. Alternatively, the two nitrogen substitutents on the tertiary amino group can be taken together to form a 3 to 7 membered ring possibly having to an additional 1 to 2 heteroatoms selected from N, O, P and S as ring vertices. Examples of amino groups include, but are not limited to, —NH₂, methylamino, ethylamino, phenylamino, N-phenyl-N-methoxyamino, dimethylamino, diethylamino, methyl-ethylamino, pyrrolidin-1-yl or piperidino etc., preferably amino, dimethylamino and diethylamino.

The term “alkylamino,” is used in its conventional sense, and refer to a secondary amino group with an alkyl substituent, and is attached to the remainder of the molecule via the nitrogen atom of the secondary amino group. Additionally, for dialkylamino groups, the alkyl portions can be the same or different and can also be combined to form a 3-7 membered ring with the nitrogen atom to which each is attached. Accordingly, a dialkylamino group is meant to include piperidinyl, pyrrolidinyl, morpholinyl, azetidinyl and the like.

The terms “alkoxy,” and “alkylthio” (or thioalkoxy) are used in their conventional sense, and refer to those alkyl groups attached to the remainder of the molecule via an oxygen atom, or a sulfur atom, respectively.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is meant to include trifluoromethyl, 2,2,2-trifluoroethyl, 4-chlorobutyl, 3-bromopropyl, and the like.

The term “amido” refers to the group —C(O)NR^{a}R^{b} or —NR^{a}C(O)R^{b}, wherein the R^{a} and R^{b} substituents are independently hydrogen, alkyl, alkenyl or aryl.

The term “boron group” as used herein, refers to the group having the general formula —BR_{c}R^{d}R^{e}, wherein R^{c}, R^{d}, and R^{e} are each an alkyl or aryl group.

The term “silicon group” as used herein, refers to the group having the general formula —SiR^{f}R^{g}R^{h}, where R^{f}, R^{g}, and R^{h} are independently an H, an alkyl group, an alkenyl group, an alkynyl group, an aryl group, or a combination of such groups.

The term “phosphorus group” as used herein, refers to an organic phosphorus group, such as for example, phosphine, phosphinite, phosphate, phosphonate, phosphate, phosphine oxide, and phosphinate, among others.

The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms. Similarly, “alkenylene” and “alkynylene” refer to the unsaturated forms of “alkylene” having double or triple bonds, respectively.

The term “heteroalkylene,” by itself or in combination with another term, means, unless otherwise stated, a stable branched or straight chain divalent radical derived from an heteroalkane and consisting of the stated number of carbon atoms and from one to five heteroatoms selected from the group consisting of O, N, Si and S, and wherein the nitrogen and sulfur atoms may optionally be oxidized and the nitrogen heteroatom may optionally be quaternized. Examples of heteroalkylene groups include —CH₂CH₂OCH₂CH₂OCH₂CH₂O—, —CH₂CH₂NHCH₂CH₂—, and the like.

The term “arylene” or “heteroarylene,” by itself or in combination with another term means, unless otherwise stated, a divalent radical derived from a C₆₋₁₄ aromatic or C₅₋₁₃ heteroaromatic ring system that is optionally substituted with 1 to 2 C₁₋₆ alkyl or C₁₋₆ heteroalkyl groups. The arylene or heteroarylene group can be covalently attached to another molecule directly through a carbon atom on the aromatic or heteroaromatic ring, or can be covalently attached to another molecule through a carbon atom or heteroatom (if present) on the C₁₋₆ alkyl or C₁₋₆ heteroalkyl substituents on the aromatic or heteroaromatic ring.

As used herein, the cyclopropenylidene-metal complex represented by the substructure,

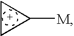

in Formula II is meant to encompass the cyclopropenylidene as a cationic,

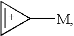

or neutral,

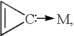

ligand.

### General

Prior to the present invention, cyclopropenylidenes of Formula I and cyclopropenylidene-metal complexes of Formula II that were stable and isolable were unknown. The inventors have described the present invention in two publications which are incorporated herein by reference for all purposes. See, Bertrand, G. et al. Science 2006, 312, 722-724; Bertrand, G. et al. Angew. Chem. Int. Ed. 2006, 45, 6652-6655.

The cyclopropenylidenes of Formula I and cyclopropenylidene-metal complexes of Formula II described herein can be incorporated into transition metal catalysts to tune the reactivity of the catalyst. Transition metal complexes comprising cyclopropenylidenes of Formula I can be useful for catalyzing a variety of synthetic organic reactions including amine arylation reactions, Suzuki coupling reactions (aryl-aryl or aryl-alkyl coupling reactions), and α-arylation reactions. Still other reactions that can benefit from the above-noted complexes include, for example, hydroformylation (of alkenes and alkynes), hydrosilylation (of alkenes, alkynes, ketones and aldehydes), metathesis (olefin (RC, CM, ROM, ROMp) ene-yne), carbonylation, hydroarylation and hydroamination.

The cyclopropenylidenes of Formula I and the cyclopropenylidene-metal complexes of Formula II having group I and II metals can function as organocatalysts similar to their N-heterocyclic carbenes (NHCs) counterpart to catalyze a wide range of organic reactions including condensation reactions, transesterification/acylation reactions, ring-opening reactions, 1,2-addition reactions, as described by Nolan, S. P. et al. Angew. Chem. Int. Ed. 2007, 46, 2-15, the teaching of which is incorporated herein by reference for all purposes.

The cyclopropenylidenes of Formula I or Ia, or of metal-complexes thereof of Formula II and IIa, can possess one or more chiral stereocenters and can exhibit planar or axial chirality.

### A. CYCLOPROPENYLIDENES OF FORMULA I

In one aspect, the present invention provides for isolable, stable cyclopropenylidenes compounds of Formula I:

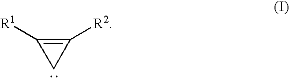

In Formula I, R¹ and R² are each members independently selected from the group consisting of amino, aryl, heteroaryl, C₁₋₁₀ alkoxy, C₂₋₁₀ alkenyloxy, C₂₋₁₀ alkynyloxy, C₁₋₁₀ alkyl, C₃₋₁₀ cycloalkyl, C₂₋₁₀ alkenyl, C₃₋₁₀ cycloalkenyl, C₂₋₁₀ alkynyl, halogen, aryloxy, heteroaryloxy, C₂₋₁₀ alkoxycarbonyl, C₁₋₁₀ alkylthio, C₂₋₁₀ alkenylthio, C₂₋₁₀ alkynylthio, C₁₋₁₀ alkylsulfonyl, C₁₋₁₀ alkylsulfinyl, aryl-C₁₋₁₀ alkyl, heteroaryl-C₁₋₁₀ alkyl, aryl-C₁₋₁₀ heteroalkyl, heteroaryl-C₁₋₁₀ heteroalkyl, a phosphorus group, a silicon group and a boron group; wherein R¹ and R² are optionally combined to form a 5- to 8-membered carbocyclic or heterocyclic ring. The aliphatic or aromatic portions of R¹ and R² are optionally independently substituted with from 1 to 4 substituents selected from the group consisting of halogen, cyano, nitro, C₁₋₄ alkyl, C₂₋₆ alkenyl, C₂₋₆ alkynyl, aryl, C₁₋₆ alkoxy, C₂₋₆ alkenyloxy, C₂₋₆ alkynyloxy, aryloxy, C₂₋₆ alkoxycarbonyl, C₁₋₆ alkylthio, C₁₋₆ alkylsulfonyl, C₁₋₆ alkylsulfinyl, oxo, imino, thiono, primary amino, carboxyl, C₁₋₆ alkylamino, C₁₋₆ dialkylamino, amido, nitrogen heterocycles, hydroxy, thiol and phosphorus groups.

In one embodiment, isolable and stable carbenes of Formula I have the Subformula Ia

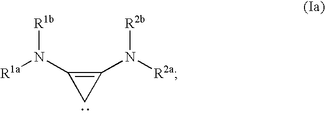

in which R^{1a}, R^{1b}, R^{2a} and R^{2b} are each members independently selected from the group consisting of hydrogen, C₁₋₁₀ alkyl, C₃₋₁₀ cycloalkyl, C₂₋₁₀ alkenyl, C₃₋₁₀ cycloalkenyl, C₂₋₁₀ alkynyl, C₁₋₁₀ heteroalkyl, C₁₋₁₀ cycloheteroalkyl, C₆₋₁₀ aryl, C₅₋₉ heteroaryl, C₆₋₁₀ aryl-C₁₋₁₀ cycloalkyl and C₅₋₉ heteroaryl-C₁₋₁₀ alkyl. Optionally, the R^{1a} and R^{1b}, R^{1b} and R^{2b}, or R^{2a} and R^{2b} groups together with the nitrogen atom to which they are attached are independently combined to form a 3- to 7-membered ring optionally having an additional 1 to 2 heteroatoms selected from N, O, P and S as ring vertices. The aliphatic or aromatic portions of R^{1a}, R^{1b}, R^{2a} and R^{2b} are independently optionally substituted with from 1 to 4 substituents selected from the group consisting of halogen, cyano, nitro, C₁₋₄ alkyl, C₂₋₆ alkenyl, C₂₋₆ alkynyl, aryl, C₁₋₆ alkoxy, C₂₋₆ alkenyloxy, C₂₋₆ alkynyloxy, aryloxy, C₂₋₆ alkoxycarbonyl, C₁₋₆ alkylthio, C₁₋₆ alkylsulfonyl, C₁₋₆ alkylsulfinyl, oxo, imino, thiono, amino, carboxyl, C₁₋₆ alkylamino, C₁₋₆ dialkylamino, amido, nitrogen heterocycles, hydroxy, thiol and phosphorus groups.

In certain aspects of this embodiment, R^{1a}, R^{1b}, R^{2a} and R^{2b} in Subformula Ia are each members independently selected from the group consisting of hydrogen, C₁₋₁₀ alkyl, C₃₋₁₀ cycloalkyl, C₆₋₁₀ aryl and C₅₋₉ heteroaryl. Optionally, the R^{1a} and R^{1b}, R^{1b} and R^{2b}, or R^{2a} and R^{2b} groups are independently combined with the nitrogen atom to which they are attached to form a 3- to 7-membered ring optionally having an additional 1 to 2 heteroatoms selected from N, O, P and S as ring vertices. The aliphatic and aromatic portions of R^{1a}, R^{1b}, R^{2a} and R^{2b} are optionally substituted with from 1 to 4 substituents selected from the group consisting of fluoro, chloro, bromo, iodo, cyano, nitro, C₁₋₄ alkyl, aryl, C₁₋₆ alkoxy, aryloxy, C₂₋₆ alkoxycarbonyl, C₁₋₆ alkylthio, C₁₋₆ alkylsulfonyl, C₁₋₆ alkylsulfinyl, oxo, imino, thiono, primary amino, carboxyl, C₁₋₆ alkylamino, C₁₋₆ dialkylamino, amido, nitrogen heterocycles, hydroxy, thiol and phosphorus groups.

In other aspects of this embodiment, R^{1a}, R^{1b}, R^{2a} and R^{2b} in Subformula Ia are selected from the group consisting of methyl, ethyl, n-propyl, iso-propyl, n-butyl, sec-butyl, iso-butyl, tert-butyl, n-pentyl, n-hexyl, cyclopentyl, cyclohexyl, norbornyl, adamantyl, phenyl, naphthyl and pyridyl. In one embodiment, R^{1a}, R^{1b}, R^{2a} and R^{2b} in Subformula Ia are each iso-propyl.

In another embodiment, in compounds of Formula I, R¹ and R² are combined to form a 5- to 8-membered carbocyclic or heterocyclic ring, wherein the heterocyclic ring can have from 1-3 heteroatoms selected from N, O, S and P. In another embodiment, in compounds of Formula Ia, R^{1b} and R^{2b} are combined to form a 5- to 8-membered carbocyclic or heterocyclic ring, wherein the heterocyclic ring can have from 1-3 heteroatoms selected from N, O, S and P. In certain aspects of the above embodiments, the 5- to 8-membered carbocyclic or heterocyclic ring can be further fused with another C₆₋₁₀ aryl or C₅₋₁₀ heteroaryl ring, wherein the C₆₋₁₀ aryl or C₅₋₁₀ heteroaryl ring can have additional substitutents as described for the R¹ and R² groups in Formula I. In one specific embodiment, R^{1b} and R^{2b} are combined to form a 5- to 8-membered heterocyclic ring, that is fused with a benzene or pyridine ring.

In one embodiment, the cyclopropenylidenes of Formula I or Subformula Ia are crystalline solids.

In another aspect, the present invention provides for a stable, isolable carbene compound comprising a plurality (i.e. from 2 to 8) of cyclopropenylidene units of Formula I or Subformula Ia, wherein each cyclopropenylidene unit is covalently attached to one or two other cyclopropenylidene units through the R¹ and/or R² groups on Formula I, or through the NR^{1a}R^{1b} and/or NR^{2a}R^{2b} groups in Subformula Ia via linking group(s) to form a stable, isolable carbene compound comprising a plurality of cyclopropenylidene groups of Formula I. In certain embodiments, the carbene compound comprising a plurality of cyclopropenylidene units of Formula I or Subformula Ia is a cyclic compound. The linking group is selected from the group consisting of C₁₋₁₀ alkylene, C₁₋₁₀ heteroalkylene, C₂₋₁₀ alkenylene, C₂₋₁₀ alkynylene, arylene and heteroarylene and is optionally substituted with 1 to 4 substituents selected from the group consisting of oxo, amino, imino, hydroxy, thiol, C₁₋₆ alkyl, C₂₋₁₀ alkenyl, C₂₋₁₀ alkynyl, C₁₋₆ alkoxy, C₂₋₁₀ alkenyloxy, C₂₋₁₀ alkynyloxy, thiono and aryl.

### B. CYCLOPROPENYLIDENES-METAL COMPLEXES OF FORMULA II

In another aspect, the present invention provides for stable, isolable cyclopropenylidene-metal complexes, wherein the metal is selected from group 1, 2-4, 6-9, 11-12 and 14-16 of the periodic table metal, and the cyclopropenylidene is of Formula I or Ia. One of skilled in the art will appreciate that such complexes can have a variety of geometries (e.g., trigonal, square planar, trigonal bipyrimidal and the like) depending on the nature of the metal and its oxidation state, and other factors including, for example, additional ligands.

In a specific embodiment, the present invention provides stable and isolable cyclopropenylidene-metal complexes of Formula II:

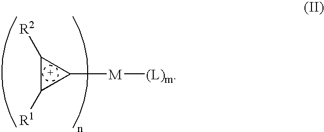

The symbol M, in Formula II, represents a metal ion. The metal ion in Formula II can be any main group metal or a transition metal. The metal ion can be a metal selected from the groups 1, 2-4, 6-9, 11-12 and 14-16 of the periodic table. In one embodiment, the metal ion can be a metal selected from the groups 1, 2 and 14-16 of the periodic table. In one preferred embodiment, the metal ion is a main group metal selected from groups 1 and 2 of the periodic table. In certain aspects of this embodiment, the main group metal is preferably, Li, Mg, Be, Na, K, Ca, Rb, Sr, Cs, Ba; or more preferably the main group metal is Li, Mg, Be and Na. In another preferred embodiment, the main group metal is Li and Mg. In another embodiment, the metal ion in Formula II, is a transition metal selected from groups 3-4, 6-9, 11-12 of the periodic table. In another embodiment, the transition metal is selected from group 11 of the periodic table. In certain aspects of this embodiment, the transition metal is Ag, Cu or Au. In one embodiment the transition metal is Ag. In another embodiment the transition metal is Cu. In another embodiment the transition metal is Au.

In Formula II, the symbols R¹ and R² are each members independently selected from the group consisting of amino, aryl, heteroaryl, C₁₋₁₀ alkoxy, C₂₋₁₀ alkenyloxy, C₂₋₁₀ alkynyloxy, C₁₋₁₀ alkyl, C₃₋₁₀ cycloalkyl, C₂₋₁₀ alkenyl, C₃₋₁₀ cycloalkenyl, C₂₋₁₀ alkynyl, halogen, aryloxy, heteroaryloxy, C₂₋₁₀ alkoxycarbonyl, C₁₋₁₀ alkylthio, C₂₋₁₀ alkenylthio, C₂₋₁₀ alkynylthio, C₁₋₁₀ alkylsulfonyl, C₁₋₁₀ alkylsulfinyl, aryl-C₁₋₁₀ alkyl, heteroaryl-C₁₋₁₀ alkyl, aryl-C₁₋₁₀ heteroalkyl, heteroaryl-C₁₋₁₀ heteroalkyl, a phosphorus group, a silicon group and a boron group; wherein R¹ and R² are optionally combined to form a 5- to 8-membered carbocyclic or heterocyclic ring. The aliphatic or aromatic portions of R¹ and R² are optionally substituted with from 1 to 4 substituents selected from the group consisting of halogen, cyano, nitro, C₁₋₄ alkyl, C₂₋₆ alkenyl, C₂₋₆ alkynyl, aryl, C₁₋₆ alkoxy, C₂₋₆ alkenyloxy, C₂₋₆ alkynyloxy, aryloxy, C₂₋₆ alkoxycarbonyl, C₁₋₆ alkylthio, C₁₋₆ alkylsulfonyl, C₁₋₆ alkylsulfinyl, oxo, imino, thiono, primary amino, carboxyl, C₁₋₆ alkylamino, C₁₋₆ dialkylamino, amido, nitrogen heterocycles, hydroxy, thiol and phosphorus groups.

In Formula II, the symbol L represents a ligand that can be present and which coordinate to the metal ion on Formula II, to render the cyclopropenylidene-metal complex stable and isolable. The ligand (L) can be a neutral ligand, anionic ligand, or an electron donating ligand. In one embodiment, the ligand (L) is an anionic ligand. In another embodiment, the ligand (L) is a neutral ligand. In yet another embodiment, the ligand (L) is a electron donating ligand. In Formula II, the subscript m is an integer from 0 to 6. In one embodiment, the subscript m is an integer from 0 to 5. In another embodiment, the subscript m is an integer from 0 to 4. In another embodiment, the subscript m is an integer from 0 to 3.

Anionic ligands suitable as additional ligands are preferably halide, pseudohalide, tetraphenylborate, perhalogenated tetraphenylborate, tetrahaloborate, hexahalophosphate, hexahaloantimonate, trihalomethanesulfonate, alkoxide, carboxylate, tetrahaloaluminate, tetracarbonylcobaltate, hexahaloferrate(III), tetrahaloferrate(III) or/and tetrahalopalladate(II), alkylsulfonate, arylsulfonate and perchlorate. Preferably, an anionic ligand is selected from halide, pseudohalide, tetraphenylborate, perfluorinated tetraphenylborate, tetrafluoroborate, hexafluorophosphate, hexafluoroantimonate, trifluoromethanesulfonate, alkoxide, carboxylate, tetrachloroaluminate, tetracarbonylcobaltate, hexafluoroferrate (III), tetrachloroferrate(III), tetrachloropalladate(II), alkylsulfonate, arylsulfonate and perchlorate. Preferred pseudohalides are cyanide, thiocyanate, cyanate, isocyanate and isothiocyanate. Neutral or electron-donor ligands suitable as additional ligands can be, for example, amines, imines, phosphines, phosphites, carbonyl compounds, alkenyl compounds (e.g., allyl compounds), carboxyl compounds, nitriles, alcohols, ethers, thiols or thioethers.

In one embodiment, the anionic ligand is selected from the group including, but not limited to, fluoride, chloride, bromide, iodide, tetrafluoroborate, tetraphenylborate, tetraperfluorophenylborate, tosylate, mesylate, triflate, acetate, trifluoroacetate, hexafluorophosphorus, hexafluoroantimony, perchlorate and the like. In another embodiment, the anionic ligand is chloride, fluoride, bromide, iodide, tetraphenylborate, tetrafluoroborate, hexafluorophosphorus and hexafluoroantimony, perchlorate. In certain preferred embodiments, the anionic ligand is tetrafluoroborate, tetraphenylborate, chloride bromide, fluoride, iodide and perchlorate.

The cyclopropenylidene-metal complex of Formula II can exist as a cationic or neutral metal complex. In one embodiment, a complex of Formula II is a neutral complex.

In another embodiment, a complex of Formula II is a cationic complex. Moreover, cationic cyclopropenylidene complexes of Formula II optionally further comprise at least one substantially non-coordinating counter-anion as necessary to form an electrically neutral cyclopropenylidene-metal complex that is stable and isolable. In one embodiment, the cyclopropenylidene-metal complex of Formula II is a cationic complex and further comprises at least one counter-anion.

Suitable counter-anions for the cyclopropenylidene-metal complex of Formula II include, halide, cyanide, tetraphenylborate, perhalogenated tetraphenylborate, tetrahaloborate, hexahalophosphate, hexahaloantimonate, trihalomethanesulfonate, arylsulfonate, alkylsulfonate, alkoxide, carboxylate, tetrahaloaluminate, tetracarbonylcobaltate, hexahaloferrate(III), tetrahaloferrate(III) and tetrahalopalladate(II), perchlorate, and the like.

In one embodiment, the counter-anion is selected from the group including, but not limited to, fluoride, chloride, bromide, iodide, tetrafluoroborate, tetraphenylborate, tetraperfluorophenylborate, tosylate, mesylate, triflate, acetate, trifluoroacetate, hexafluorophosphorus, hexafluoroantimony, perchlorate and the like. In another embodiment, the counter-anion is chloride, fluoride, bromide, iodide, tetraphenylborate, tetrafluoroborate, hexafluorophosphorus and hexafluoroantimony, perchlorate. In certain preferred embodiments, the counter-anion is tetrafluoroborate, tetraphenylborate, chloride and perchlorate.

In Formula II, the subscript n is an integer from 1 to 8. In preferred embodiments, the subscript n is an integer from 1 to 6, or from 1 to 4, or from 1 to 3, or from 1 to 2.

When the metal complex of Formula II has a plurality of cyclopropenylidene units (i.e., the subscript n is an integer from 2 to 8), each cyclopropenylidene units is optionally covalently attached to one or two other cycloproprenyl units through the R¹ and/or R² groups located on each cyclopropenylidene unit using linking group(s) to form a multi-dentate cyclopropenylidene ligand (e.g., a bi-, tri-, tetra-dentate ligand). In certain embodiments, a multi-dentate cyclopropenylidene ligand is a cyclic multi-dentate ligand. The linking group is selected from the group consisting of C₁₋₁₀ alkylene, C₁₋₁₀ heteroalkylene, C₂₋₁₀ alkenylene, C₂₋₁₀ alkynylene, arylene and heteroarylene. The linking group is optionally substituted with 1 to 4 substituents selected from the group consisting of oxo, amino, imino, hydroxy, thiol, C₁₋₆ alkyl, C₂₋₁₀ alkenyl, C₂₋₁₀ alkynyl, C₁₋₆ alkoxy, C₂₋₁₀ alkenyloxy, C₂₋₁₀ alkynyloxy, thiono and aryl.

In one embodiment, the cyclopropenylidene-metal complex of Formula II has Subformula Ia:

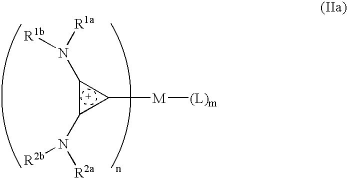

in which the symbols R^{1a}, R^{1b}, R^{2a} and R^{2b} are each members independently selected from the group consisting of hydrogen, C₁₋₁₀ alkyl, C₃₋₁₀ cycloalkyl, C₂₋₁₀ alkenyl, C₃₋₁₀ cycloalkenyl, C₂₋₁₀ alkynyl, C₁₋₁₀ heteroalkyl, C₁₋₁₀ cycloheteroalkyl, 6- to 10-membered aryl and 5- to 9-membered heteroaryl. Optionally, the R^{1a} and R^{1b}, R^{1b} and R^{2b}, or R^{2a} and R^{2b} groups together with the nitrogen atom to which they are attached are independently combined to form a 3- to 7-membered ring optionally having an additional 1 to 2 heteroatoms selected from N, O, P and S as ring vertices. The aliphatic or aromatic portions of R^{1a}, R^{1b}, R^{2a} and R^{2b} are independently optionally substituted with from 1 to 4 substituents selected from the group consisting of halogen, cyano, nitro, C₁₋₄ alkyl, C₂₋₆ alkenyl, C₂₋₆ alkynyl, aryl, C₁₋₆ alkoxy, C₂₋₆ alkenyloxy, C₂₋₆ alkynyloxy, aryloxy, C₂₋₆ alkoxycarbonyl, C₁₋₆ alkylthio, C₁₋₆ alkylsulfonyl, C₁₋₆ alkylsulfinyl, oxo, imino, thiono, amino, carboxyl, C₁₋₆ alkylamino, C₁₋₆ dialkylamino, amido, nitrogen heterocycles, hydroxy, thiol and phosphorus groups. There is the proviso that in Formula Ia, when R^{1a}, R^{1b}, R^{2a} and R^{2b} are each isopropyl, M is lithium and the subscript n is 1, then the counter-anion is not perchlorate or tetraphenylborate. In certain aspects of this embodiment, R^{1a}, R^{1b}, R^{2a} and R^{2b} in Subformula Ia are each members independently selected from the group consisting of hydrogen, C₁₋₁₀ alkyl, C₃₋₁₀ cycloalkyl, C₆₋₁₀ aryl and C₅₋₉ membered heteroaryl. Optionally, the R^{1a} and R^{1b}, R^{1b} and R^{2b}, R^{2a} and R^{2b} groups are independently combined with the nitrogen atom to which they are attached to form a 3- to 7-membered ring optionally having an additional 1 to 2 heteroatoms selected from N, O, P and S as ring vertices. The aliphatic and aromatic portions of R^{1a}, R^{1b}, R^{2a} and R^{2b} are optionally substituted with from 1 to 4 substituents selected from the group consisting of fluoro, chloro, bromo, iodo, cyano, nitro, C₁₋₄ alkyl, aryl, C₁₋₆ alkoxy, aryloxy, C₂₋₆ alkoxycarbonyl, C₁₋₆ alkylthio, C₁₋₆ alkylsulfonyl, C₁₋₆ alkylsulfinyl, oxo, imino, thiono, primary amino, carboxyl, C₁₋₆ alkylamino, C₁₋₆ dialkylamino, amido, nitrogen heterocycles, hydroxy, thiol and phosphorus groups.

In other aspects of this embodiment, R^{1a}, R^{1b}, R^{2a} and R^{2b} in Subformula Ia are selected from the group consisting of methyl, ethyl, n-propyl, iso-propyl, n-butyl, sec-butyl, iso-butyl, tert-butyl, n-pentyl, n-hexyl, cyclopentyl, cyclohexyl, norbornyl, adamantyl, phenyl, naphthyl and pyridyl. In one embodiment, R^{1a}, R^{1b}, R^{2a} and R^{2b} in Subformula IIa are each iso-propyl.

In Subformula IIa, the symbols M and L, and the subscripts m and n are as described for Formula II.

In one preferred embodiment of the invention, R^{1a}, R^{1b}, R^{2a} and R^{2b} in Subformula IIa are each iso-propyl; the subscript n is 1; and M is Li or Ag. In another preferred embodiment, R^{1a}, R^{1b}, R^{2a} and R^{2b} in Subformula IIa are each iso-propyl; the subscript n is 2; and M is Mg or Ag.

In one embodiment, Subformula IIa has the structure selected from the group consisting of:

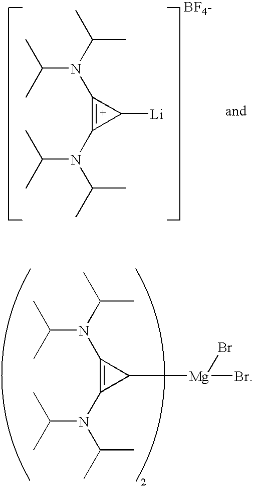

When the metal complex of Subformula IIa has a plurality of cyclopropenylidene units (i.e., the subscript n is an integer from 2 to 8), the cyclopropenylidene units optionally can be covalently attached to each other through the R^{1a}, R^{1b}, R^{2a} and R^{2b} groups located on each cyclopropenylidene unit using linking group(s) to form a multi-dentate cyclopropenylidene ligand (e.g., a bi-, tri-, tetra-dentate ligand). In certain embodiments, a multi-dentate cyclopropenylidene ligand is a cyclic multi-dentate ligand. The linking group is selected from the group consisting of C₁₋₁₀ alkylene, C₁₋₁₀ heteroalkylene, C₂₋₁₀ alkenylene, C₂₋₁₀ alkynylene, arylene and heteroarylene. The linking group is optionally substituted with 1 to 4 substituents selected from the group consisting of oxo, amino, imino, hydroxy, thiol, C₁₋₆ alkyl, C₂₋₁₀ alkenyl, C₂₋₁₀ alkynyl, C₁₋₆ alkoxy, C₂₋₁₀ alkenyloxy, C₂₋₁₀ alkynyloxy, thiono and aryl.

In addition, although Formula II and Formula IIa each illustrates one resonance structure of the cyclopropenium cation, it is noted that other resonance structures exist and all are within the scope of the invention.

### C. PREPARATION OF COMPOUNDS

In another aspect, the present invention provides for a method of making isolable, stable cyclopropenylidenes of Formulas I and Subformula Ia, and isolable, stable cyclopropenylidene-metal complexes of Formula II and Subformula Ia.

Stable and isolable cyclopropenylidenes of Formula I or Subformula Ia or cyclopropenylidene-metal complexes of Formula II or Subformula Ia can be prepared by various methods, including thermolysis or photolysis of the corresponding diazo precursors; desulfurization of 1-thiocarbonyl cyclopropenium precursors; deprotonation of cyclopropenium precursors, metal-halogen exchange on halo-cyclopropenium precursors; and desilylation of silyl-cyclopropenium precursors, among others. In certain embodiments stable and isolable cyclopropenylidenes (Formula I and Subformula Ia) and metal chelates thereof (Formula II and Subformula IIa), are preferably prepared by the methods illustrated in Scheme 1. In Scheme 1, R, R′ and R″ each represent a non-interfering substituent, M represents a metal ion, and X⁻ represents a compatible counter-anion, which is preferably a counter-anion that does not degrade or otherwise affect the stability of the cyclopropenium precursor (i.e., i, ii, iii) or the stable, and isolable cyclopropenylidene (i.e., iv) or cyclopropenylidene-metal (i.e., v) product.

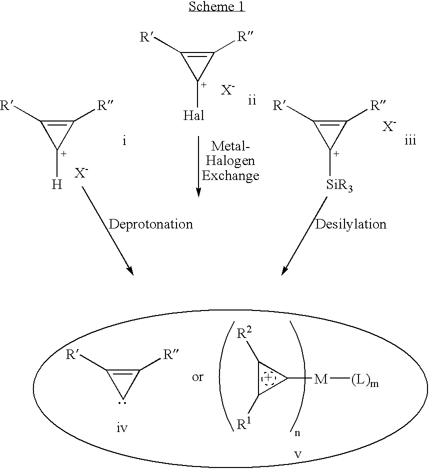

In one method, the removal of the hydrogen atom (e.g., by deprotonation) of the corresponding conjugate acid, i.e., the cyclopropenium salt of i, can produce stable and isolable cyclopropenylidene compounds of Formula iv or Formula v. In another method, stable, isolable carbene compound Iv or v are prepared by reacting a halocyclopropenium salt with a reagent capable of undergoing a metal-halogen exchange reaction (e.g., t-butyl lithium, alkylMgBr, Li and Mg metal). In yet another method, isolable, stable carbenes iv or v are prepared by treatment of a silylcyclopropenium salt iii with a desilylating reagent (e.g., KF, tetrabutylammonium fluoride).

One advantage of the methods described in Scheme 1 is their rapidity, even at lower temperatures. Moreover, for the cyclopropenium salts, i.e., i, ii, iii, can be prepared in large quantities and are generally thermally stable. For example, protonated cyclopropenium salts i can be prepared as described by Komatsu, K. et al. which is incorporated herein by reference for all purposes. (See, Komatsu, K. et al. Chem. Rev. 103, 1371 (2003). Halo-cyclopropenium salts ii can be prepared as described by Yoshida, Z., which is incorporated herein by reference for all purposes. (See, Yoshida, Z., Curr. Chem. 40, 47 (1973).) Silyl-cyclopropenium salt iii can be prepared as described by Weiss, R. et al., which is incorporated herein by reference for all purposes. (See, Weiss, R. et al. Angew. Chem. Intl. Ed. Engl. 18, 1979, 473.)

The methods described above, can produce either the neutral (non-coordinated) cyclopropenylidene product iv or the metal chelated product v. If a cyclopropenylidene iv that is not coordinated to a metal ion is the desired product, then in the synthetic preparation of iv, it is preferable to select reagents that does not contain metal ions that would coordinate to the cyclopropenylidene product. For example, to form a stable, isolable cyclopropenylidene iv by deprotonation of cyclopropenium salt i, it is preferable use a Brönsted base having a counter-cation which substantially does not coordinate to, or otherwise interact (e.g., ionically, covalently, etc.), with the cyclopropenylidene product iv. The counter-cation to the base can be an organic or inorganic cation. Suitable counter-cations that substantially do not coordinate to the cyclopropenylidene product iv include, but are not limited to, an alkali metal or alkali earth metal from rows 3, 4, 5, 6 and 7. Examples of such counter-cations include potassium, sodium cesium, calcium, barium, strontium, rubidium, and combinations thereof.

Along similar lines, if a cyclopropenylidene-metal complex v is the desired product, then in the synthetic preparation of v, it is preferable to choose reagents that comprises metal cations that can coordinate to the cyclopropenylidene product. For example, to form a stable, isolable cyclopropenylidene-metal complex v by deprotonation of cyclopropenium salt i, it is preferable use a Brönsted base having a counter-cation(s) which can coordinate to, the cyclopropenylidene product. Suitable counter-cations for this purpose can include alkali metal or alkali earth metals from rows 2 and 3 of the periodic table, preferably lithium and magnesium. Similarly, to form a stable, isolable cyclopropenylidene-metal complex v by metal-halogen exchange, it is preferable to choose a metal-halogen exchange reagent which contains a metal ion that can coordinate to the cyclopropenylidene product (e.g., t-Butyl Lithium, Phenyl-MgBr),

As described above, the cyclopropenium cations i, ii, iii each has a counter-anion X⁻ that is compatible with the cyclopropenium cations i, ii, iii and/or the cyclopropenylidene products iv, v (i.e., X⁻ does not substantially degrade or adversely affect the stability of a cyclopropenium cation or a cyclopropenylidene product once formed). Examples of suitable counterions X⁻ include halide, cyanide, tetraphenylborate, perhalogenated tetraphenylborate, tetrahaloborate, hexahalophosphate, hexahaloantimonate, trihalomethanesulfonate, arylsulfonate, alkylsulfonate, alkoxide, carboxylate, tetrahaloaluminate, tetracarbonylcobaltate, hexahaloferrate(III), tetrahaloferrate(III) and tetrahalopalladate(II), and the like.

In a specific embodiment, the present invention provides for a method of making a isolable, stable carbene of Formula I, the method comprising: contacting a cyclopropenium salt of Formula III with a Brönsted base under conditions suitable to form the isolable, stable carbene of Formula I;

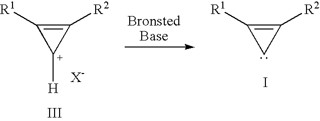

wherein in Formulae I and/or III, R¹ and R² are each independently selected from the group consisting of amino, aryl, heteroaryl, C₁₋₁₀ alkoxy, C₂₋₁₀ alkenyloxy, C₂₋₁₀ alkynyloxy, C₁₋₁₀ alkyl, C₃₋₁₀ cycloalkyl, C₂₋₁₀ alkenyl, C₃₋₁₀ cycloalkenyl, C₂₋₁₀ alkynyl, halogen, aryloxy, heteroaryloxy, C₂₋₁₀ alkoxycarbonyl, C₁₋₁₀ alkylthio, C₂₋₁₀ alkenylthio, C₂₋₁₀ alkynylthio, C₁₋₁₀ alkylsulfonyl, C₁₋₁₀ alkylsulfinyl, a phosphorus group, a silicon group and a boron group; wherein the aliphatic or aromatic portions of R¹ and R² are optionally substituted with from 1 to 4 substituents selected from the group consisting of halogen, cyano, nitro, C₁₋₄ alkyl, C₂₋₆ alkenyl, C₂₋₆ alkynyl, aryl, C₁₋₆ alkoxy, C₂₋₆ alkenyloxy, C₂₋₆ alkynyloxy, aryloxy, C₂₋₆ alkoxycarbonyl, C₁₋₆ alkylthio, C₁₋₆ alkylsulfonyl, C₁₋₆ alkylsulfinyl, oxo, imino, thiono, primary amino, carboxyl, C₁₋₆ alkylamino, C₁₋₆ dialkylamino, amido, nitrogen heterocycles, hydroxy, thiol and phosphorus groups; X is a counter-anion; and the Brönsted base is associated with, and thus comprises, an alkali metal or alkali earth metal cation selected from rows 3, 4, 5 and 6 of the periodic table.

In preferred embodiments, a Brönsted base is associated with a cation preferably selected from rows 3, 4, and 5 of the periodic table, and even more preferably from rows 3 and 4 of the periodic table. In one embodiment, the cation component to a Brönsted base is preferably potassium, sodium, magnesium, cesium, calcium or barium. In one embodiment, the cation associated with a Brönsted base is preferably sodium or potassium. In another embodiment, the cation is preferably potassium. Examples of specific Brönsted bases that are suitable for use for the deprotonation cyclopropenium III to form isolable stable cyclopropenylidenes I, include but are not limited to, potassium bis(trimethylsilyl)amide, sodium bis(trimethylsilyl)amide, potassium hydride, sodium hydride, sodium and potassium alkoxides (e.g., sodium methoxide, sodium tert-butoxide, potassium tert-butoxide), sodium and potassium aryloxides and derivatives thereof. In one embodiment, the Brönsted base of is selected from the group consisting of potassium bis(trimethylsilyl)amide, sodium bis(trimethylsilyl)amide, potassium hydride and sodium hydride.

In Formula III, in one embodiment, the counter-anion X⁻ is preferably, fluoride, chloride, bromide, iodide, tetrafluoroborate, tetraphenylborate, tetraperfluorophenylborate, tosylate, mesylate, triflate, acetate, trifluoroacetate, hexafluorophosphorus, hexafluoroantimony and the like. In another embodiment, X⁻ is preferably chloride, fluoride, bromide, iodide, tetraphenylborate, tetrafluoroborate, perchlorate, hexafluorophosphorus and hexafluoroantimony. In another embodiment, X⁻ is a counter-anion selected from the group consisting of X— is a counter-anion selected from the group consisting of halide, cyanide, tetraphenylborate, perhalogenated tetraphenylborate, tetrahaloborate, trihalomethanesulfonate, arylsulfonate, alkylsulfonate, alkoxide, carboxylate and perchlorate. In yet another embodiment, X— is a counter-anion is selected from the group consisting of chloride, fluoride, bromide, iodide, tetraphenylborate, tetrafluoroborate and perchlorate. In certain preferred embodiments, X is preferably tetrafluoroborate, tetraphenylborate or chloride.

The following examples are meant to illustrate but not limit the present invention.

### D. EXAMPLES

All manipulations were performed under an inert atmosphere of argon using standard Schlenk techniques. Dry, oxygen-free solvents were employed. ¹H and ¹³C NMR spectra were recorded on Varian Inova 300, 400, 500 and Brucker 300 spectrometers. The abbreviations KHMDS=potassium bis(trimethylsilyl)amide or potassium hexamethyldisilylazide; and Et₂O=ethyl ether.

### Example 1

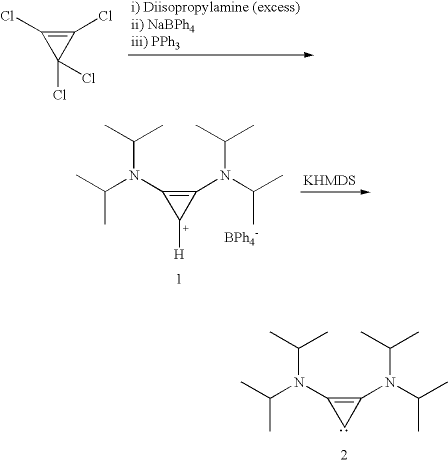

Synthesis of Bis(diisopropylamino)cyclopropenium tetraphenylborate 1: This compound has been prepared by combination, with slight modifications, of two procedures previously reported (see, R. Weiss, C. Priesner, Angew. Chem. Int. Ed. Engl. 17, 445 (1978); Z. Yoshida, Y. Tawara, J Am. Chem. Soc. 93, 2574 (1971)) and using tetraphenylborate as a counter-anion.

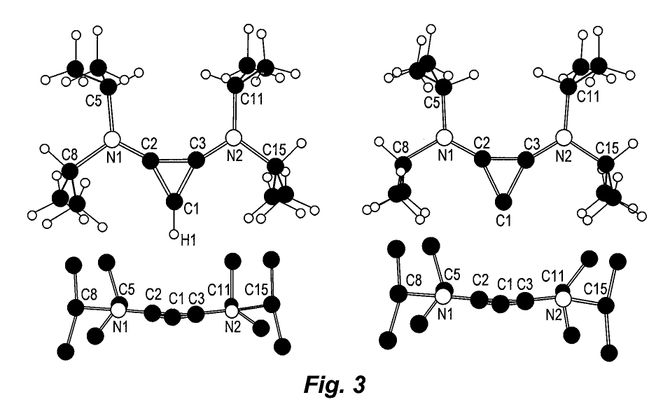

Diisopropylamine (22.03 g, 0.220 mol) was added dropwise at 0° C. to a stirred solution oftetrachlorocyclopropene (7.745 g, 0.043 mol) in CH₂Cl₂ (300 ml). After 6 hours at 0° C., the solution was warmed to room temperature and sodium tetraphenylborate (14.90 g, 0.043 mol) was added. The suspension was stirred overnight, and then refluxed for 4 hours. After cooling down the solution to room temperature, triphenylphosphine (11.42 g, 0.043 mol) was added. Immediately after addition of the triphenylphosphine, water (200 ml) was added and the mixture was stirred overnight with a vent to open air. The organic layer was separated and washed with water (3×500 ml). Then the organic phase was dried with anhydrous MgSO₄ and concentrated under vacuum. After washing with pentane (200 ml), and drying under vacuum, the cyclopropenium salt 1 was obtained as a pale yellow solid (21.82 g, 90%). Recrystallization in CH₂Cl₂/Et₂O at −20° C. afforded 1 as yellow crystals. FIG. 3 (left side) illustrates the ORTEP views of 1: (m.p. 142-144° C.); ¹H NMR (CDCl₃, 25° C., 300 MHz): 7.43-7.46 (m, 8H, CHₐᵣ), 7.02-7.07 (m, 8H, CHₐᵣ), 6.87-6.92 (m, 4H, CHₐᵣ), 4.92 (s, 1H, CH), 3.76 (sept, J=6.8, 2H, CHCH₃), 3.57 (sept, J=6.8, 2H, CHCH₃), 1.22 (d, J=6.8, 12H, CHCH₃), 1.11 (d, J=6.8, 12H, CHCH₃), ¹³C NMR (CDCl₃, 25° C., 100 MHz): 164.35 (q, J=49.2, CHₐᵣ), 136.35 (CHₐᵣ), 133.16 (C_{ring}), 125.66 (CHₐᵣ), 121.81 (CHₐᵣ), 99.71 (CH_{ring}), 55.63 (CHCH₃), 50.03 (CHCH₃), 21.08 (CHCH₃), 20.87 (CHCH₃).

Synthesis of Bis(diisopropylamino)cyclopropenylidene 2: A 1:1 mixture of the cyclopropenium salt 1 (4.50 g, 8.08 mmol) and potassium bis(trimethylsilyl)amide (1.61 g, 8.08 mmol) was cooled to −78° C., and Et₂O (60 ml) was slowly added. The suspension was stirred for 10 min. and then warmed up to room temperature. After evaporation of the solvent under vacuum, hexane (60 ml) was added and the suspension was stirred for 10 min. After filtration, the yellow solution was kept at −20° C. overnight to give 2 as yellow crystals (0.38 g, 20%). FIG. 3 (right side) illustrates the molecular ORTEP view of cyclopropenylidene 2: (m.p. 107-109° C.); ¹H NMR (C₆D₆, 25° C., 400 MHz): 3.65 (sept, J=6.4, 4H, CHCH₃), 1.24 (broad s, 24H, CHCH₃); ¹³C NMR (C₆D₆, 25° C., 100 MHz): 185.51 (C_{carb}), 158.97 (C), 51.32 (CHCH₃), 21.99 (CHCH₃).

### Example 2

The following example provides X-ray crystallographic data for compounds 1 and 2.

Crystal Structure Determination of compounds 1 and 2: The Bruker X8-APEX (Bruker (2005). APEX 2 version 2.0-2. Bruker AXS Inc., Madison, Wis., U.S.A.) X-ray diffraction instrument with Mo-radiation was used for data collection of compounds 1c and 1. All data frames were collected at low temperatures (T=100 K) using an ω, φ-scan mode (0.3° ω-scan width, hemisphere of reflections) and integrated using a Bruker SAINTPLUS software package (Bruker (2005). SAINT version V7.21A. Bruker AXS Inc., Madison, Wis., U.S.A.). The intensity data were corrected for Lorentzian polarization. Absorption corrections were performed using the SADABS program (Bruker (2004). SADABS version 2004/1. Bruker Analytical X-Ray System, Inc., Madison, Wis., U.S.A.). The SIR97 (Altornare, A., et al. SIR 97 (1999) J. Appl. Cryst. 32, 115-122) was used for direct methods of phase determination, and Bruker SHELXTL software package (Bruker, (2003). SHELXTL Software Version 6.14, Dec, BRuker Analytical X-Ray System, Inc., Madison, Wis., U.S.A.) for structure refinement and difference Fourier maps. Atomic coordinates, isotropic and anisotropic displacement parameters of all the non-hydrogen atoms of two compounds were refined by means of a full matrix least-squares procedure on F². All H-atoms were included in the refinement in calculated positions riding on the C atoms, with U[iso] fixed at 20% higher than isotropic parameters of carbons atoms which they were attached. Drawings of molecules were performed using Ortep 3 (ORTEP3 for Windows—L, J. Farrugia, J. Appl. Crystallogr. 1997, 30, 565.) and POVRay for Windows (POV-Ray for Windows Version 3.6 Persistence of Vision Raytracer Pty. Ltd.).

Crystal and structure parameters of 1: size 0.47×0.76×0.79 mm³, monoclinic, space group C2/c, a=37.490(5) Å, b=10.4373(14) Å, c=20.168(3) Å, a=90.0°, β=107.850(2)° γ=90.00, V=7511.7(17) Å³, ρ^{calcd}=1.195 Mg/m³, 2θₘₐₓ=52.80°, Mo-radiation (λ=0.71073 Å), low temperature=100(2) K, total reflections collected=27045, independent reflections=7577 (Rᵢₙₜ=0.0630, R_{sig}=0.0723), 4632 (61.2%) reflections were greater than 2σ(I), index ranges—46<=h<=46, −12<=k<=12, 25<=1<=18 absorption coefficient μ=0.274 mm⁻¹; max/min transmission=0.9811 and 0.8821, 451 parameters were refined and converged at R1=0.0481, wR2=0.0981, with intensity I>2σ (I), the final difference map was 0.279 and −0.379 e.A⁻³.

Crystal and structure parameters of 2: size 0.21×0.15×0.06 mm³, orthorhombic, space group Pna2(1), a=11.7506(17) Å, b=7.5798(11) Å, c=17.329(2) Å, α=β=γ=90.0°, V=1543.4(4) Å³, ρ^{calcd}=1.017 g/cm³, 2θₘₐₓ=55.02°, Mo-radiation (λ=0.71073 Å), low temperature=100(2) K, reflections collected=11258, independent reflections=2235 (Rᵢₙₜ=0.0770, R_{sig}=0.0561), 1761 (78.8%) reflections were greater than 2σ (I), index ranges—15<=h<=15, −9<=k<=6, −21<=1<=8, absorption coefficient μ=0.059 mm⁻¹; max/min transmission=0.9964 and 0.9876, 163 parameters were refined and converged at R1=0.0457, wR2=0.0913, with intensity 1>2σ (I), the final difference map was 0.168 and −0.159 e.Å⁻³.

### Example 3

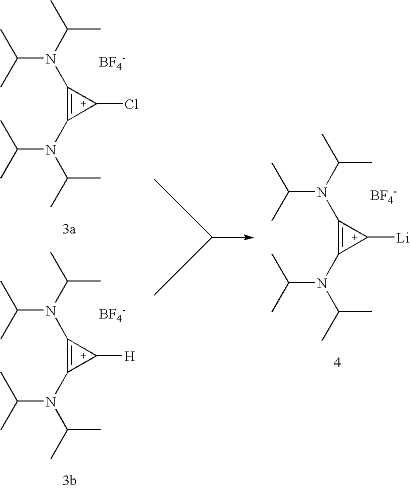

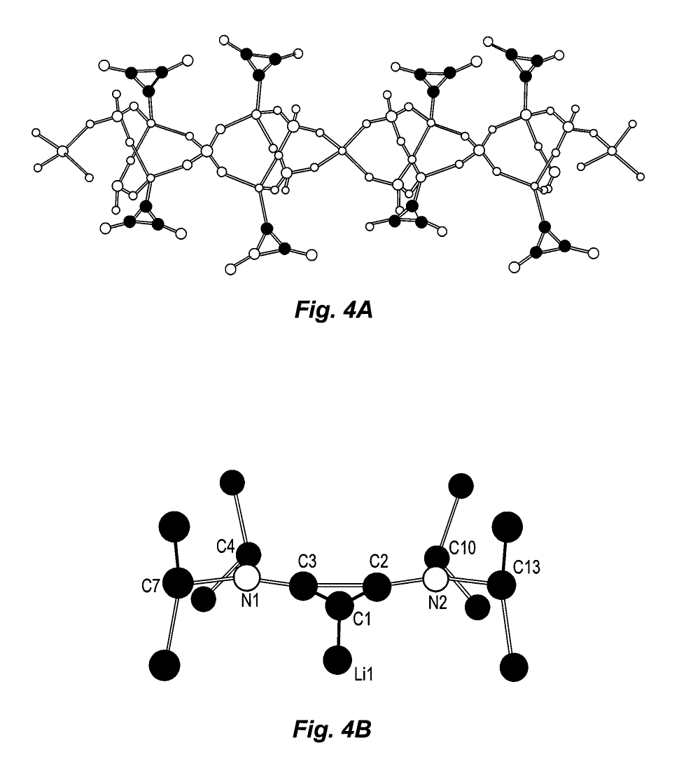

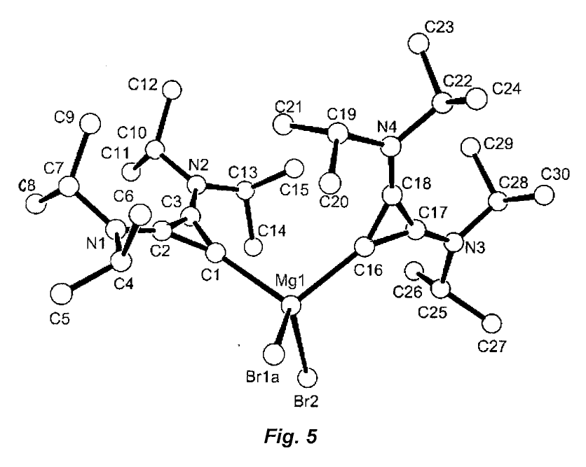

Synthesis of Bis(diisopropylamino)cyclopropenylidene Lithium Adduct (4): Compounds 3a and 3b were prepared by methods described in the literature with the exception that NaBF4 was used for the anion exchange: 3a, 30.0 g, 90%, m.p. 120-121° C.; 3b, 33.5 g, 90%, m.p. 121-122° C. See, Bertrand, G. et al. Science 2006, 312, 733; and Yoshida, Z. et al. J. Am. Chem. Soc. 1972, 93, 2574. n-Butyl lithium (1 equiv.) was added to a suspension of 3a (3.00 g, 8.36 mmol) or 3b (3.00 g, 9.25 mmol) in diethyl ether (80 mL) at −78 C. The reaction was stirred for 15 minutes and then warmed to room temperature. After concentration in a vacuum and washing with hexane (40 mL), a white powder was obtained. The residue was recrystallized from a solution of diethyl ether at −25° C. and colorless crystals were obtained (yielded: 1.59 g (45%) from 3a, 1.88 g (48%) from 3b. The X-ray crystal structure of 4 in the solid state is shown in FIG. 4A. The molecular ORTEP view of 4 is shown in FIG. 4B: M.p. 88-90° C. (decomp); ¹H NMR (C₆H₆, 25° C., 300 MHz): δ=3.62 (brm, 4H, CH), 1.21 ppm (brm, 24H, CH₃); ¹³CNMR ([D₈]THF, 25° C., 75 Mhz): δ=166.8 (CLi), 154.9 (C_{ring}) 50.4 (br, CH), 21.1 ppm (CH₃); ⁷LiNMR ([D₈]THF, 25° C., 233 Mhz) δ 0.27 ppm (rel. to LiCl in D₂O as external standard.

### Example 4

### Synthesis of Cyclopropenylidene-Magnesium Complex 6

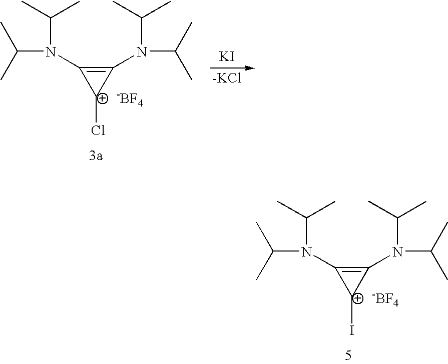

Step 1: A 500 ml round bottom flask was loaded with chlorocyclopropenium salt 3a (10 g, 27.88 mmol) and KI (4.63 g, 27.88 mmol). The mixture was covered with 250 ml of acetone and stirred for 2 days at rt. The acetone was removed by rotovap and the residue was washed with water (3×100 ml). The organic residue was dried under high vacuum to give iodocyclopropenium salt 5 (11.92 g, 95%): ¹H NMR (CD₂Cl₂, 25° C., 600 MHz): 4.05 (sept, J=6.8, 2H, CHCH₃), 3.92 (sept, J=6.8, 2H, CHCH₃), 1.47 (d, J=6.8, 12H, CHCH₃), 1.33 (d, J=6.8, 12H, CHCH₃), ¹³C NMR (CD₂Cl₂, 25° C., 100 MHz): 139.76 (C_{ring}), 64.71 (CI), 57.63 (CHCH₃), 49.69 (CHCH₃), 22.72 (CHCH₃), 21.28 (CHCH₃).

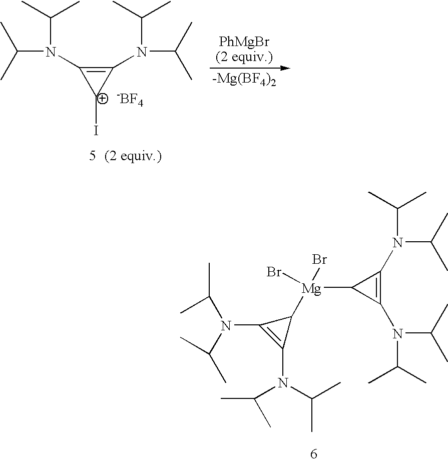

Step 2: To a suspension of the iodocyclopropenium salt 5 (1 g, 450.10 mmol) in diethyl ether (40 ml) a solution of PhMgBr (1 M, 4.55 ml) was added at rt. The suspension was stirred for thirty minutes and then filtered and set in a freezer at −20° C. overnight where colorless crystals of magnesium cyclopropenylidene complex 6 formed (461 mg, 70%): ¹H NMR ([D₈]THF, 25° C., 300 MHz): δ=4.25-4.05 (brm, 2H, CH), 3.65-3.30 (brm, 2H, CH), 1.85-1.65 ppm (brm, 12H, CH₃), 1.15-0.75 ppm (brm, 12H, CH₃); ¹³CNMR ([D₈]THF, 25° C., 75 MHz): δ=170.8 (CMg), 150.3 (C_{ring}), 51.4 (br, CH), 21.5 ppm (CH₃).

### Example 5

The following example provides crystallographic data and structure refinement of bis(diisopropylaminocyclopropenylidene)-magnesium complex 6.

| Empirical formula | C₃₄H₆₄Br₂MgN₄O | |
|---|---|---|
| Formula weight | 729.02 | |
| Temperature | 100(2) K | |
| Wavelength | 0.71073 Å | |
| Crystal system | Orthorhombic | |
| Space group | P2(1)2(1)2(1) | |
| Unit cell dimensions | a = 10.8857(16) Å | α = 90° |
| | b = 10.9503(16) Å | β = 90° |
| | c = 33.017(5) Å | γ = 90° |

| | |
|---|---|
| Volume | 3935.7(10) Å³ |
| Z | 4 |
| Density (calculated) | 1.230 Mg/m³ |
| Absorption coefficient | 2.105 mm⁻¹ |
| F(000) | 1544 |
| Crystal size | 0.28 × 0.19 × 0.09 mm³ |
| Theta range for data collection | 1.23 to 21.97° |
| Index ranges | −11 <= h <= 11, −11 <= k <= 11, |
| | −34 <= 1 <= 34 |
| Reflections collected | 19655 |
| Independent reflections | 4780 [R(int) = 0.0330] |
| Completeness to theta = 21.97° | 99.0% |
| Absorption correction | Sadabs |
| Max. and min. transmission | 0.8267 and 0.5853 |
| Refinement method | Full-matrix least-squares on F² |
| Data/restraints/parameters | 4780/6/401 |
| Goodness-of-fit on F² | 1.201 |
| Final R indices [I > 2sigma(I)] | R1 = 0.0756, wR2 = 0.1736 |
| R indices (all data) | R1 = 0.0774, wR2 = 0.1746 |
| Absolute structure parameter | 0.85(3) |
| Extinction coefficient | 0.0017(3) |
| Largest diff. peak and hole | 1.042 and −1.189 e.Å⁻³ |

What is claimed is:

1. An isolable, stable carbene of Formula I: 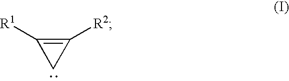
wherein
R¹ and R² are each members independently selected from the group consisting of amino, aryl, heteroaryl, C₁₋₁₀ alkoxy, C₂₋₁₀ alkenyloxy, C₂₋₁₀ alkynyloxy, C₁₋₁₀ alkyl, C₃₋₁₀ cycloalkyl, C₂₋₁₀ alkenyl, C₃₋₁₀ cycloalkenyl, C₂₋₁₀ alkynyl, halogen, aryloxy, heteroaryloxy, C₂₋₁₀ alkoxycarbonyl, C₁₋₁₀ alkylthio, C₂₋₁₀ alkenylthio, C₂₋₁₀ alkynylthio, C₁₋₁₀ alkylsulfonyl, C₁₋₁₀ alkylsulfinyl, aryl-C₁₋₁₀ alkyl, heteroaryl-C₁₋₁₀ alkyl, aryl-C₁₋₁₀ heteroalkyl, heteroaryl-C₁₋₁₀ heteroalkyl, a phosphorus group, a silicon group and a boron group; wherein R¹ and R² are optionally combined to form a 5- to 8-membered carbocyclic or heterocyclic ring; wherein the aliphatic or aromatic portions of R¹ and R² are optionally substituted with from 1 to 4 substituents selected from the group consisting of halogen, cyano, nitro, C₁₋₄ alkyl, C₂₋₆ alkenyl, C₂₋₆ alkynyl, aryl, C₁₋₆ alkoxy, C₂₋₆ alkenyloxy, C₂₋₆ alkynyloxy, aryloxy, C₂₋₆ alkoxycarbonyl, C₁₋₆ alkylthio, C₁₋₆ alkylsulfonyl, C₁₋₆ alkylsulfinyl, oxo, imino, thiono, primary amino, carboxyl, C₁₋₆ alkylamino, C₁₋₆ dialkylamino, amido, nitrogen heterocycles, hydroxy, thiol and phosphorus groups.

2. The compound of claim 1, wherein said isolable, stable carbene is of Subformula Ia: 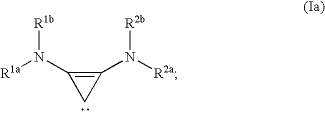
wherein R^{1a}, R^{1b}, R^{2a} and R^{2b} are each independently selected from the group consisting of hydrogen, C₁₋₁₀ alkyl, C₃₋₁₀ cycloalkyl, C₂₋₁₀ alkenyl, C₃₋₁₀ cycloalkenyl, C₂₋₁₀ alkynyl, C₁₋₁₀ heteroalkyl, C₁₋₁₀ cycloheteroalkyl, C₆₋₁₀ aryl, C₅₋₉ heteroaryl, C₆₋₁₀ aryl-C₁₋₁₀ cycloalkyl and C₅₋₉ heteroaryl-C₁₋₁₀ alkyl, wherein the R^{1a} and R^{1b}, R^{1b} and R^{2b}, or R^{2a} and R^{2b} groups together with the nitrogen atom to which they are attached are independently optionally combined to form a 3- to 7-membered ring optionally having an additional 1 to 2 heteroatoms selected from N, O, P and S as ring vertices, and wherein the aliphatic or aromatic portions of R^{1a}, R^{1b}, R^{2a} and R^{2b} are independently optionally substituted with from 1 to 4 substituents selected from the group consisting of halogen, cyano, nitro, C₁₋₄ alkyl, C₂₋₆ alkenyl, C₂₋₆ alkynyl, aryl, C₁₋₆ alkoxy, C₂₋₆ alkenyloxy, C₂₋₆ alkynyloxy, aryloxy, C₂₋₆ alkoxycarbonyl, C₁₋₆ alkylthio, C₁₋₆ alkylsulfonyl, C₁₋₆ alkylsulfinyl, oxo, imino, thiono, amino, carboxyl, C₁₋₆ alkylamino, C₁₋₆ dialkylamino, amido, nitrogen heterocycles, hydroxy, thiol and phosphorus groups.

3. The compound of claim 2, wherein R^{1a}, R^{1b}, R^{2a} and R^{2b} are each independently selected from the group consisting of hydrogen, C₁₋₁₀ alkyl, C₃₋₁₀ cycloalkyl, C₆₋₁₀ aryl and C₅₋₉ heteroaryl, wherein R^{1a} and R^{1b}, R^{1b} and R^{2b}, or R^{2a} and R^{2b} are optionally independently combined with the nitrogen atom to which they are attached to form a 3- to 7-membered ring optionally having an additional 1 to 2 heteroatoms selected from N, O, P and S as ring vertices; and wherein the aliphatic and aromatic portions of R^{1a}, R^{1b}, R^{2a} and R^{2b} are optionally substituted with from 1 to 4 substituents selected from the group consisting of fluoro, chloro, bromo, iodo, cyano, nitro, C₁₋₄ alkyl, aryl, C₁₋₆ alkoxy, aryloxy, C₂₋₆ alkoxycarbonyl, C₁₋₆ alkylthio, C₁₋₆ alkylsulfonyl, C₁₋₆ alkylsulfinyl, oxo, imino, thiono, primary amino, carboxyl, C₁₋₆ alkylamino, C₁₋₆ dialkylamino, amido, nitrogen heterocycles, hydroxy, thiol and phosphorus groups.

4. The compound of claim 3, wherein R^{1a}, R^{1b}, R^{2a} and R^{2b} are selected from the group consisting of methyl, ethyl, n-propyl, iso-propyl, n-butyl, sec-butyl, iso-butyl, tert-butyl, n-pentyl, n-hexyl, cyclopentyl, cyclohexyl, norbornyl, adamantyl, phenyl, naphthyl and pyridyl.

5. The compound of claim 4, wherein R^{1a}, R^{1b}, R^{2a} and R^{2b} are each iso-propyl.

6. The compound of claim 1, wherein said isolable, stable carbene is a crystalline solid.

7. An isolable, stable cyclopropenylidene-metal complex comprising a cyclopropenylidene of Formula I or Ia that is coordinated to a metal ion selected from group 1-4, 6-9, 11-12 and 14-16 of the periodic table.

8. An isolable, stable cyclopropenylidene-metal complex of Formula II: 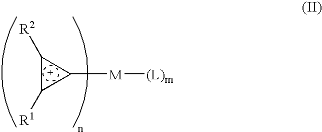
wherein R¹ and R² are each members independently selected from the group consisting of amino, aryl, heteroaryl, C₁₋₁₀ alkoxy, C₂₋₁₀ alkenyloxy, C₂₋₁₀ alkynyloxy, C₁₋₁₀ alkyl, C₃₋₁₀ cycloalkyl, C₂₋₁₀ alkenyl, C₃₋₁₀ cycloalkenyl, C₂₋₁₀ alkynyl, halogen, aryloxy, heteroaryloxy, C₂₋₁₀ alkoxycarbonyl, C₁₋₁₀ alkylthio, C₂₋₁₀ alkenylthio, C₂₋₁₀ alkynylthio, C₁₋₁₀ alkylsulfonyl, C₁₋₁₀ alkylsulfinyl, aryl-C₁₋₁₀ alkyl, heteroaryl-C₁₋₁₀ alkyl, aryl-C₁₋₁₀ heteroalkyl, heteroaryl-C₁₋₁₀ heteroalkyl, a phosphorus group, a silicon group and a boron group, wherein R¹ and R² are optionally combined to form a 5- to 8-membered carbocyclic or heterocyclic ring; wherein the aliphatic or aromatic portions of R¹ and R² are optionally substituted with from 1 to 4 substituents selected from the group consisting of halogen, cyano, nitro, C₁₋₄ alkyl, C₂₋₆ alkenyl, C₂₋₆ alkynyl, aryl, C₁₋₆ alkoxy, C₂₋₆ alkenyloxy, C₂₋₆ alkynyloxy, aryloxy, C₂₋₆ alkoxycarbonyl, C₁₋₆ alkylthio, C₁₋₆ alkylsulfonyl, C₁₋₆ alkylsulfinyl, oxo, imino, thiono, primary amino, carboxyl, C₁₋₆ alkylamino, C₁₋₆ dialkylamino, amido, nitrogen heterocycles, hydroxy, thiol and phosphorus groups;
the symbol M represents a metal ion selected from groups 1-4, 6-9, 11-12 and 14-16 of the periodic table; the subscript n is an integer from 1 to 8;
L is an anionic, neutral or electron donating ligand; the subscript m is an integer from 0 to 6; and
wherein a cyclopropenylidene-metal complex of Formula II optionally comprises at least one counter-anion.

9. The cyclopropenylidene-metal complex of claim 8, wherein M represents a metal selected from group 1 and 2 of the periodic table.

10. The cyclopropenylidene-metal complex of claim 8, wherein M represents a metal selected from group 11 of the periodic table.

11. The cyclopropenylidene-metal complex of claim 8, wherein the subscript n is an integer from 1 to 2.

12. The cyclopropenylidene-metal complex of claim 8, wherein L represents a ligand selected from the group consisting of halide, pseudohalide, tetraphenylborate, perhalogenated tetraphenylborate, tetrahaloborate, hexahalophosphate, hexahaloantimonate, trihalomethanesulfonate, alkoxide, carboxylate, tetrahaloaluminate, tetracarbonylcobaltate, hexahaloferrate(III), tetrahaloferrate(III), tetrahalopalladate(II), alkylsulfonate, arylsulfonate, perchlorate, cyanide, thiocyanate, cyanate, isocyanate isothiocyanate, amines, imines, phosphines, phosphites, carbonyl compounds, alkenyl compounds, allyl compounds, carboxyl compounds, nitrites, alcohols, ethers, thiols and thioethers.

13. The cyclopropenylidene-metal complex of claim 8 having the Subformula IIa: 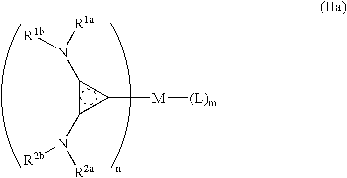
wherein R^{1a}, R^{1b}, R^{2a} and R^{2b} are each members independently selected from the group consisting of hydrogen, C₁₋₁₀ alkyl, C₃₋₁₀ cycloalkyl, C₂₋₁₀ alkenyl, C₃₋₁₀ cycloalkenyl, C₂₋₁₀ alkynyl, C₁₋₁₀ heteroalkyl, C₁₋₁₀ cycloheteroalkyl, C₆₋₁₀ aryl, C₅₋₉ heteroaryl, C₆₋₁₀ aryl-C₁₋₁₀ cycloalkyl and C₅₋₉ heteroaryl-C₁₋₁₀ alkyl, wherein the R^{1a} and R^{1b}, R^{1b} and R^{2b}, or R^{2a} and R^{2b} groups together with the nitrogen atom to which they are attached 9 are optionally independently combined to form a 3- to 7-membered ring optionally having an additional 1 to 2 heteroatoms selected from N, O, P and S as ring vertices; wherein the aliphatic or aromatic portions of R^{1a}, R^{1b}, R^{2a} and R^{2b} are independently optionally substituted with from 1 to 4 substituents selected from the group consisting of halogen, cyano, nitro, C₁₋₄ alkyl, C₂₋₆ alkenyl, C₂₋₆ alkynyl, aryl, C₁₋₆ alkoxy, C₂₋₆ alkenyloxy, C₂₋₆ alkynyloxy, aryloxy, C₂₋₆ alkoxycarbonyl, C₁₋₆ alkylthio, C₁₋₆ alkylsulfonyl, C₁₋₆ alkylsulfinyl, oxo, imino, thiono, amino, carboxyl, C₁₋₆ alkylamino, C₁₋₆ dialkylamino, amido, nitrogen heterocycles, hydroxy, thiol and phosphorus groups; the symbol M is a metal ion; and the subscript n is an integer from 1 to 8; the symbol L represents an anionic, neutral or electron donating ligand; the subscript m is an integer from 0 to 6; and wherein a cyclopropenylidene-metal complex of Formula IIa optionally comprises at least one counter-anion.

14. The cyclopropenylidene-metal complex of claim 13, wherein n is from 1 to 2.

15. The cyclopropenylidene-metal complex of claim 13, wherein R^{1a}, R^{1b}, R^{2a} and R^{2b} are independently selected from the group consisting of methyl, ethyl, n-propyl, iso-propyl, n-butyl, sec-butyl, iso-butyl, tert-butyl, n-pentyl, n-hexyl, cyclopentyl, cyclohexyl, norbornyl, adamantyl, phenyl, naphthyl and pyridyl.

16. The cyclopropenylidene-metal complex of claim 13, wherein M is a metal selected from group 1 and 2 of the periodic table.

17. The cyclopropenylidene-metal complex of claim 13, wherein M is Li or Mg.

18. The cyclopropenylidene-metal complex of claim 13, wherein L is a ligand selected from the group consisting of tetrafluoroborate, tetraphenylborate, chloride bromide, fluoride, iodide and perchlorate.

19. The cyclopropenylidene-metal complex of claim 13, wherein Formula II has the structure selected from the group consisting of

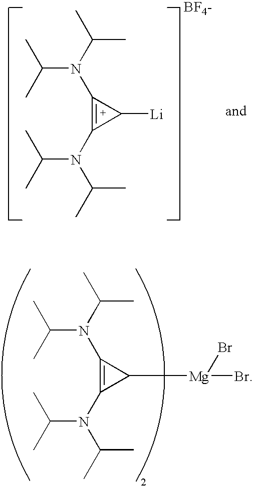

20. A method of making a isolable, stable carbene of Formula I, the method comprising: contacting a cyclopropenium salt of Formula III with a Brönsted base under conditions suitable to form the isolable, stable carbene of Formula I; 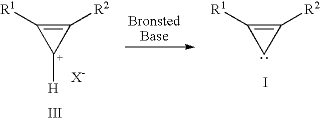
wherein in Formulae I and/or III, R¹ and R² are each independently selected from the group consisting of amino, aryl, heteroaryl, C₁₋₁₀ alkoxy, C₂₋₁₀ alkenyloxy, C₂₋₁₀ alkynyloxy, C₁₋₁₀ alkyl, C₃₋₁₀ cycloalkyl, C₂₋₁₀ alkenyl, C₃₋₁₀ cycloalkenyl, C₂₋₁₀ alkynyl, halogen, aryloxy, heteroaryloxy, C₂₋₁₀ alkoxycarbonyl, C₁₋₁₀ alkylthio, C₂₋₁₀ alkenylthio, C₂₋₁₀ alkynylthio, C₁₋₁₀ alkylsulfonyl, C₁₋₁₀ alkylsulfinyl aryl-C₁₋₁₀ alkyl, heteroaryl-C₁₋₁₀ alkyl, aryl-C₁₋₁₀ heteroalkyl, heteroaryl-C₁₋₁₀ heteroalkyl, a phosphorus group, a silicon group and a boron group, wherein R¹ and R² are optionally combined to form a 5- to 8-membered carbocyclic or heterocyclic ring; wherein the aliphatic or aromatic portions of R¹ and R² are optionally substituted with from 1 to 4 substituents selected from the group consisting of halogen, cyano, nitro, C₁₋₄ alkyl, C₂₋₆ alkenyl, C₂₋₆ alkynyl, aryl, C₁₋₆ alkoxy, C₂₋₆ alkenyloxy, C₂₋₆ alkynyloxy, aryloxy, C₂₋₆ alkoxycarbonyl, C₁₋₆ alkylthio, C₁₋₆ alkylsulfonyl, C₁₋₆ alkylsulfinyl, oxo, imino, thiono, primary amino, carboxyl, C₁₋₆ alkylamino, C₁₋₆ dialkylamino, amido, nitrogen heterocycles, hydroxy, thiol and phosphorus groups;
X is a counter-anion; and
the Brönsted base comprises an alkali metal or alkali earth metal cation selected from rows 3, 4, 5 and 6 of the periodic table.

21. The method of claim 20, wherein said Brönsted base comprises a cation is selected from the group consisting of potassium, sodium, magnesium, cesium, calcium and barium.

22. The method of claim 21, wherein said base comprises a cation selected from potassium and sodium.

23. The method of claim 22, wherein said Brönsted base of is selected from the group consisting of potassium bis(trimethylsilyl)amide, sodium bis(trimethylsilyl)amide, potassium hydride and sodium hydride, potassium tert-butoxide, sodium tert-butoxide.

24. The method of claim 20, wherein in Formula III, X⁻ is a counter-anion selected from the group consisting of halide, cyanide, tetraphenylborate, perhalogenated tetraphenylborate, tetrahaloborate, trihalomethanesulfonate, arylsulfonate, alkylsulfonate, alkoxide, carboxylate and perchlorate.

25. The method of claim 24, wherein X⁻ is a counter-anion is selected from the group consisting of chloride, fluoride, bromide, iodide, tetraphenylborate, tetrafluoroborate and perchlorate.
